(19) United States
(12) Patent Application Publication
(10) Pub. No.: US 2006/0064153 A1
(43) Pub. Date: Mar. 23, 2006

(54) Intravascular stent and method for producing the stent

(75) Inventors: Lutz Langhans, Starnberg (DE); Dieter Mairhormann, Buchloe (DE); Wulf Polack, Munchen (DE); Andreas Wetzig, Munchen (DE)

Correspondence Address: LERNER GREENBERG STEMER LLP, P O BOX 2480, HOLLYWOOD, FL, 33022-2480, US

(73) Assignee: Carl Baasel Lasertechnik GmbH & Co. KG

(21) Appl. No.: 10/980,967

(22) Filed: Nov. 4, 2004

(30) Foreign Application Priority Data
Sep. 3, 2004 (DE) 10 2004 043 166.

(51) Int. Cl. A61F 2/06; B23K 26/00

(52) U.S. Cl. 623/1.15; 219/121.71

(57) ABSTRACT

An intravascular stent or a similar filigree structure is produced in one piece from a cylindrical thin-walled tube. The stent has a plurality of bridges with side faces and apertures delimited by them. At least part of a side face extends obliquely with respect to the outer and inner surface of the stent. The tube is held on a machining apparatus in such a way that it can turn about its central longitudinal axis. The apertures are cut with a laser beam directed at the outer surface of the tube. The remaining wall areas form the bridges. The beam axis of the laser beam is directed toward at least part of the length of a kerf, which has been formed in the tube wall to create an aperture, in such a way that said beam axis hits the outer surface of the tube obliquely.

### BACKGROUND OF THE INVENTION

Field of the Invention

The invention relates to an intravascular one-piece stent and to a method for producing stents and similar filigree structures, for example helical springs or interlinked chain structures with a cylindrical surface, from thin-walled thin tubes. In conventional one-piece stents, the side faces of the bridges extend at right angles with respect to the outer surface or inner surface of the stent or in the radial direction.

### SUMMARY OF THE INVENTION

It is an object of the invention to provide a stent and a method of producing the stent which overcome the disadvantages associated with the heretofore-known devices and methods of this general type and which provides for an alternatively configured stent and a method by which such stents and similar structures can be produced.

With the foregoing and other objects in view there is provided, in accordance with the invention, an intravascular stent, comprising:
a one-piece, substantially cylindrical stent body formed of a plurality of bridges with side faces, said stent body having an outer surface and an inner surface;
said stent body having a plurality of apertures delimited by said side faces of said bridges, and at least a portion of said side face extending obliquely with respect to at least one of said outer surface of said stent body and said inner surface of said stent body.

With the above and other objects in view there is also provided, in accordance with the invention, a method of producing a filigree structure, such as an intravascular stent with bridges and apertures from a thin-walled tube, which comprises the following steps:
holding the thin-walled tube on a machining apparatus for rotation thereof about a central longitudinal axis of the tube;
directing a laser beam at an outer surface of the tube, and thereby cutting apertures out of the tube wall, and forming bridges with remaining wall areas;
thereby directing a beam axis of the laser beam, at least along a part of a length of a kerf formed in the tube wall defining the aperture, to impinge obliquely on the outer surface of the tube.

In other words, the objects of the invention are achieved with an intravascular stent which is in one piece, at least part of a side face is oriented obliquely with respect to the outer surface or inner surface of the stent, i.e., not radially. Whereas the bridges of conventional stents have an approximately rectangular cross section, in the proposed stent, by contrast, the bridges have cross sections which are, for instance, trapezoidal or triangular. For example, the inclinations of the side faces can be chosen such that the outer surface of a bridge is smaller than its inner surface. Accordingly, an opening delimited by bridges widens from the inner surface of the stent to the outer surface of the stent. The fact that the side faces are not oriented radially or perpendicularly with respect to the outer surface and inner surface of the stent means that new design possibilities are generally opened up. For example, structures can be produced which are able to hook onto one another or interlock, which could be of advantage for collapsible stents, for example. It is also conceivable that the inclination or orientation of the side faces of the bridges changes in the direction of the central longitudinal axis of a stent. Likewise, the bridges can have different widths in said direction. Viewed in the radial direction, for example, a side face preferably has a planar configuration.

A method for the production of filigree structures, made up of bridges and apertures, from a thin-walled thin tube, in particular for production of stents, involves holding a tube on a machining apparatus in such a way that it can turn about its central longitudinal axis. A laser beam is directed at the outer surface of the tube and cuts apertures out from the tube wall, and the wall areas which are left form the bridges. The beam axis of the laser beam is directed toward at least part of the length of a kerf, which has been formed in the tube wall to create an aperture, in such a way that said beam axis hits the outer surface of the tube obliquely. Such an orientation can be achieved quite generally by moving the beam axis of the laser beam, by a corresponding change in position of the tube, or by a combination of these two measures. Bridges with side faces which have an intermittently or continuously changing inclination can be obtained by suitably changing the orientation of the beam axis during the cutting procedure.

In a preferred implementation of the method, the angle of incidence is moved by virtue of the fact that the laser beam, starting from a center position in which its beam axis intersects the outer surface of the tube at right angles, is moved together with an interposed focussing lens in a direction extending transverse to the central longitudinal axis. In another preferred implementation of the method, a beam axis extending obliquely with respect to the outer surface of the tube is achieved by the fact that the laser beam, starting from a center position in which its beam axis intersects the outer surface of the tube at right angles, is moved parallel to a plane containing the central longitudinal axis of the tube, and an interposed focusing lens is maintained in its original location assigned to the center position. Since the focusing lens is in a fixed position in this variant of the method, there is to this extent no need for any actuating mechanisms. The laser beam could be moved in said manner simply by optical measures.

Other features which are considered as characteristic for the invention are set forth in the appended claims.

Although the invention is illustrated and described herein as embodied in an intravascular stent and method for its production, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a diagrammatic perspective view of a stent according to the invention;

FIG. 2 is a side view of a tube used to produce a stent;

FIGS. 3A to 3E are diagrammatic views of possible ways of changing the position of a laser beam relative to a tube which is to be machined;

FIG. 4 is a partial cross-sectional view of a stent with bridge side faces that extend at right angles with respect to the stent surface, i.e. radially;

FIG. 5 is a partial cross-section, similar to FIG. 4, of a stent with bridge side faces not extending perpendicularly with respect to the outer surface of the stent; and

FIG. 6 is a partial longitudinal section showing a wall area of a stent, likewise with bridge side faces oriented obliquely with respect to the stent surface.

### DESCRIPTION OF THE PREFERRED EMBODIMENTS

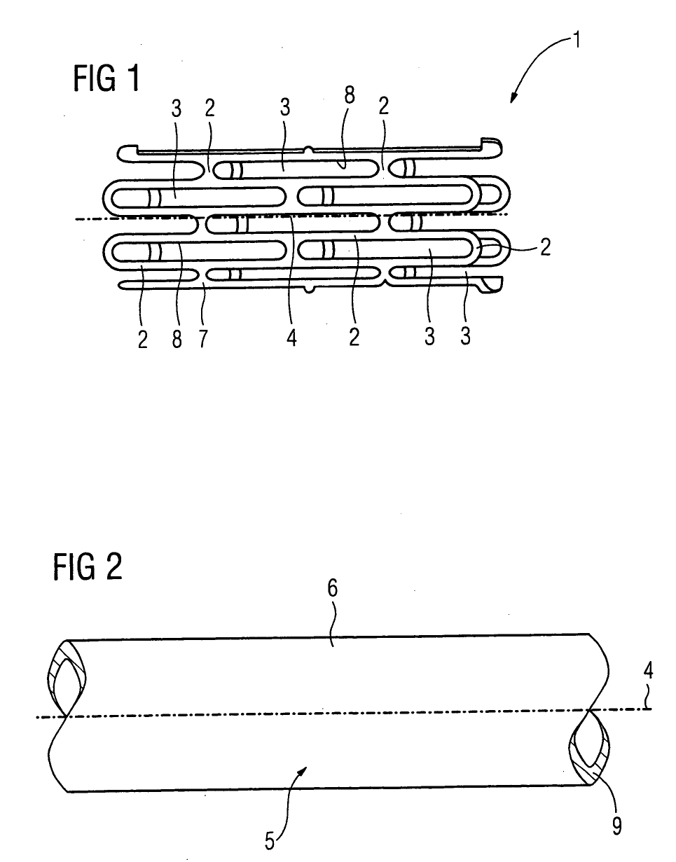

Referring now to the figures of the drawing in detail and first, particularly, to FIG. 1 thereof, there is shown a stent 1 formed of a multiplicity of integrally connected bridges 2, said bridges delimiting apertures 3. The stent 1 is in the shape of a sleeve or cylinder, i.e. the bridges 2 are arranged concentrically about a central longitudinal axis 4.

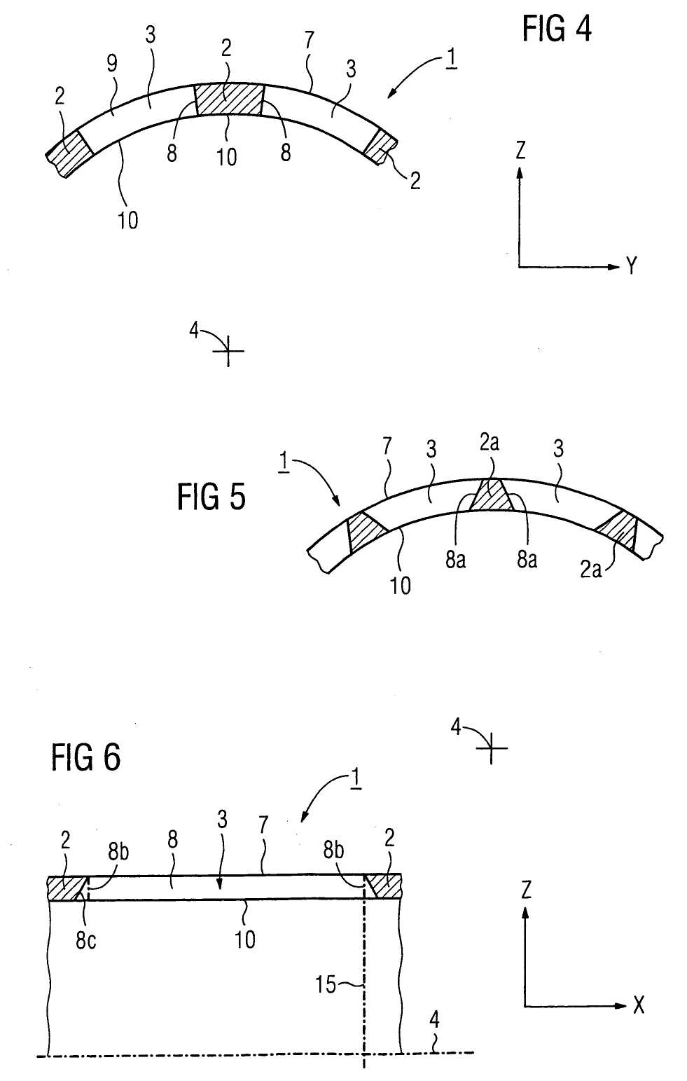

A stent of the type shown in FIG. 1 is made from a tube 5 having a wall thickness corresponding to the subsequent bridges 2. The machining is done on a non-illustrated apparatus on which the tube 5 is fixed on a holder. The holder is configured such that the tube can turn about its central longitudinal axis 4 and can move along this axis. In conventional methods and apparatus, known for example from U.S. Pat. Nos. 5,759,192; 5,780,807; 6,131,266 to Richard Saunders and European Patent EP 0 820 738 B1. The disclosures and details concerning the laser-cutting apparatus are herewith incorporated by reference. The cutting patterns for cutting out apertures 3 and producing the filigree bridge structure are created using a fixed laser beam and by moving the tube 5, that is to say rotating it and moving it along its central longitudinal axis 4. The laser beam 13 is thus oriented in such a way that its beam axis 12 hits the outer surface 6 of the tube or outer surface 7 of the subsequent stent at right angles, in other words it intersects the central longitudinal axis 4. Accordingly, all cuts generated in this way are radial cuts. In a bridge 2 produced by such a cut, its side faces 8 are oriented such that they extend at right angles with respect to the outer surface 7 of the stent and the inner surface 10 of the stent (see FIG. 4).

In a method according to the invention, a tube 5 is held in such a way that it can likewise turn about its central longitudinal axis 4 and can be moved in the direction of the axis. A machining apparatus is configured, however, in such a way that the beam axis 12 of a laser beam 13 directed at the tube 5 can be oriented at different angles with respect to the outer surface 6 of the tube. In this way it is also possible to create bridges 2 having side faces 8 which are not radial. An example of such a stent is shown in FIG. 5. Its apertures 3 widen from the inside outward. The bridges 2a have a trapezoid cross section.

Additional information concerning a novel laser cutting apparatus may be found, for example, in the commonly assigned, depending U.S. application Ser. No. ______ [atty. docket MOH-P040109] filed Oct. 15, 2004, which is herewith incorporated by reference.

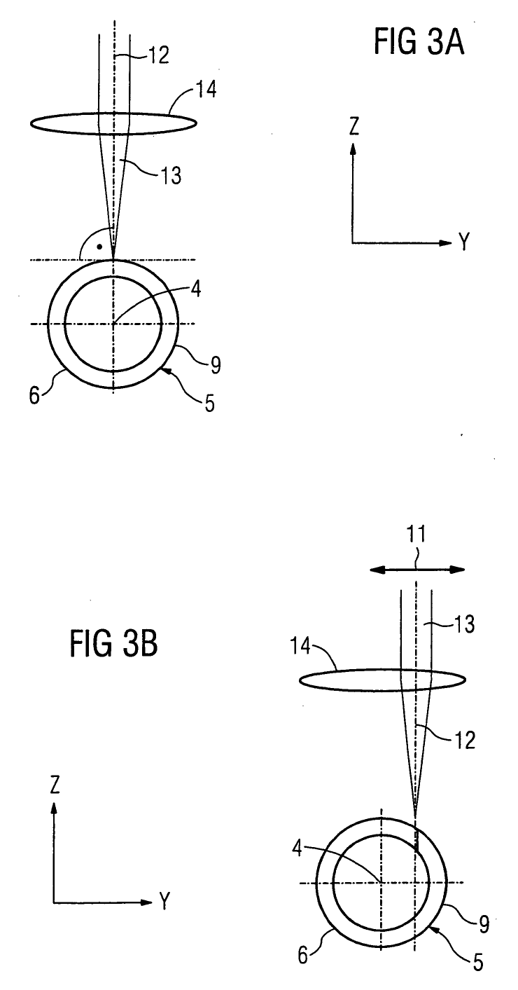

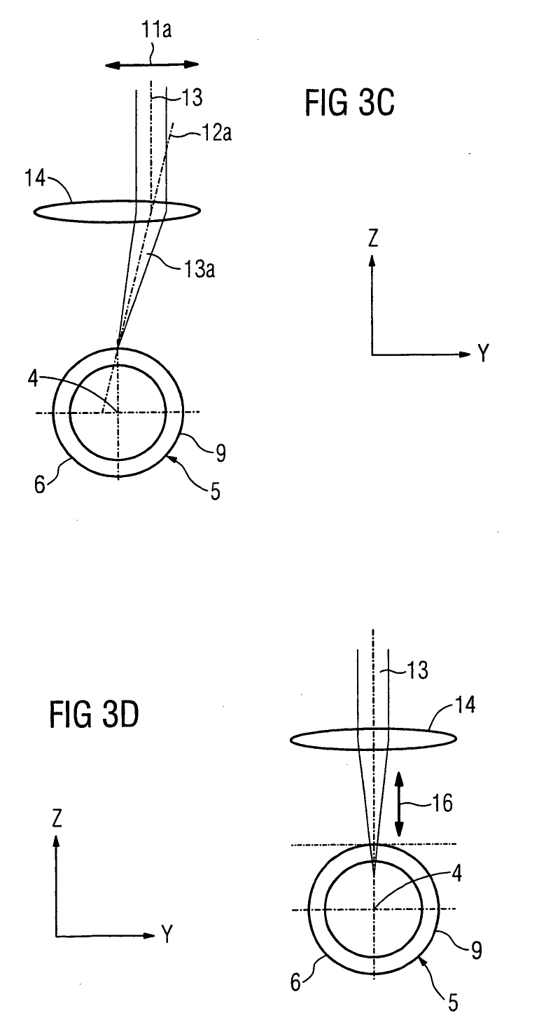

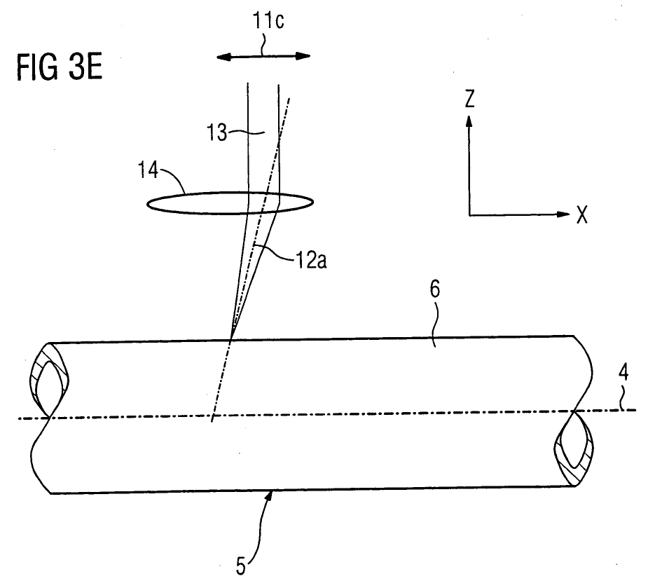

The laser beam 13 can be oriented in a center position such that its beam axis 12 extends at right angles to the outer surface 6 of the tube and intersects the central longitudinal axis 4 of the tube 5. To focus the laser beam 13, it is guided through a focusing lens 14 (see FIG. 3A). In this arrangement, and in the arrangements described below, the tube extends in a plane defined by the axes X, Y. In the center position, the laser beam 13 or its beam axis 12 extends perpendicular to the X-Y plane (Z axis).

To create side faces 8 which do not extend radially or do not intersect the outer surface 7 of the stent at right angles, the laser beam 13 is moved or deflected in various ways.

In the variant indicated in FIG. 3B, this is achieved by moving the laser beam 13, together with the focusing lens 14, at right angles with respect to the central longitudinal axis 4 of the tube 5 or parallel to the X-Y plane (double arrow 11). For example, to be able to create a bridge 2a in a stent according to FIG. 5, the position of rotation of the tube 5 and the position of the laser beam 13 in the Y direction are suitably chosen. In doing so, the lens 14 must also be moved accordingly in the Z direction.

Another possibility illustrated in FIG. 3C involves keeping the focusing lens 14 fixed in position and moving only the laser beam 13 in the Y direction, as is indicated by the double arrow 11a. The beam axis 12a of the laser beam 13a leaving the lens 14 extends obliquely with respect to the outer surface 6 of the tube. The beam axis 13 is oriented in the z direction. The beam axis 12a then extends in the Y-Z plane. With cutting in the X direction (tube 5 fixed in rotation, moved in X direction), the side faces Ba obtained are oriented obliquely with respect to the outer surface 7 of the stent (FIG. 5). With cutting in the circumferential direction or Y direction (axially fixed tube 5 is rotated), side faces 8b would be obtained (FIG. 6) which extend in the Y-Z plane. To obtain side faces 8c extending obliquely in the Y-Z plane, the beam axis 12a must have an oblique position relative to the Y-Z plane (FIG. 3E). This can be achieved, for example, with an arrangement according to FIG. 3E in which the beam axis 12a extends in the X-Z plane and the laser beam 13 is moved in the X direction (double arrow 11c) while the lens 14 is stationary.

FIG. 3D shows that different cutting thicknesses can be set by the focusing lens 14 being able to be positioned in the Z direction (double arrow 16).

All the cuts shown in FIGS. 3B, 3C and 3D can also be produced using a fixed laser beam 13 oriented as in FIG. 3A, by moving the tube 5 in the Y direction and/or pivoting it in the X-Z plane about an axis extending in the Y direction.

This application claims the priority, under 35 U.S.C. § 119, of German patent application No. 10 2004 043 166.3, filed Sep. 3, 2004; the entire disclosure of the prior application is herewith incorporated by reference.

We claim:

1. An intravascular stent, comprising:
a one-piece, substantially cylindrical stent body formed of a plurality of bridges with side faces, said stent body having an outer surface and an inner surface;
said stent body having a plurality of apertures delimited by said side faces of said bridges, and at least a portion of said side face extending obliquely with respect to at least one of said outer surface of said stent body and said inner surface of said stent body.

2. A method of producing a stent, which comprises the following steps:
holding a thin-walled tube on a machining apparatus for rotation thereof about a central longitudinal axis of the tube;
directing a laser beam at an outer surface of the tube to thereby form the stent body according to claim 1, with the apertures cut out of the tube wall and remaining wall areas defining the bridges; and
thereby directing a beam axis of the laser beam, at least along a partial length of a kerf formed in the tube wall defining the aperture, to impinge obliquely on the outer surface of the tube.

3. A method of producing a filigree structure with bridges and apertures from a thin-walled tube, which comprises the following steps:
holding the thin-walled tube on a machining apparatus for rotation thereof about a central longitudinal axis of the tube;
directing a laser beam at an outer surface of the tube, and thereby cutting apertures out of the tube wall, and forming bridges with remaining wall areas;
thereby directing a beam axis of the laser beam, at least along a part of a length of a kerf formed in the tube wall defining the aperture, to impinge obliquely on the outer surface of the tube.

4. The method according to claim 3, which comprises varying an orientation of the beam axis during creation of an aperture.

5. The method according to claim 3, which comprises defining the beam axis extending obliquely with respect to the outer surface of the tube by displacing the laser beam together with a focusing lens, from a center position in which the beam axis impinges on the outer surface of the tube perpendicularly, in a direction transverse to the central longitudinal axis of the tube and in a radial plane of the tube.

6. The method according to claim 3, which comprises defining the beam axis extending obliquely with respect to the outer surface of the tube by moving a beam axis of the laser beam, starting from a center position in which the beam axis intersects the outer surface of the tube perpendicularly, parallel to a plane containing the central longitudinal axis of the tube, and maintaining a focusing lens in an original location thereof assigned to the center position.

7. In an apparatus for producing a filigree structure, made up of bridges and apertures, from a thin-walled tube, the improvement which comprises means associated with the apparatus for orienting and positioning a laser beam in accordance with the method of claim 3.

8. In an apparatus for producing an intravascular stent, made up of bridges and apertures, from a thin-walled tube, the improvement which comprises means associated with the apparatus for orienting and positioning a laser beam in accordance with the method of claim 2.
